(19) United States
(12) Patent Application Publication
(10) Pub. No.: US 2026/0324968 A1
(43) Pub. Date: Oct. 22, 2026

(54) SYSTEM AND METHOD FOR DIAGNOSING EYE DISORDERS

(71) Applicant: Eyecuracy Ltd., Tel Aviv (IL)

(72) Inventors: Albert KASHCHENEVSKY, Tel Aviv (IL); Anatoly VOROBEY, Rishon LeTsiyon (IL)

(21) Appl. No.: 19/467,935

(22) Filed: Feb. 3, 2026

Related U.S. Application Data
(63) Continuation-in-part of application No. PCT/IL2025/050673, filed on Aug. 7, 2025.
(60) Provisional application No. 63/680,409, filed on Aug. 7, 2024.

(51) Int. Cl. A61B 3/113; A61B 3/08; G02B 27/00; G02B 27/01

(52) U.S. Cl. CPC A61B 3/113; A61B 3/08; G02B 27/0093; G02B 27/0172; G02B 2027/014

(57) ABSTRACT

System and method are presented for measuring near point of convergence (NPC) and/or convergence recovery point (CRP) of patient's eyes. An eye tracking device provides measured eye-related data indicative of visual axes of the eyes associated with eyes' vergence during measurements; a target tracking device, calibrated with respect to the eye tracking device, monitors distances from the eyes to a moving target, onto which the eyes are to be focused, and provides measured target-related data indicative of a target location relative to the eyes; and a processing circuitry analyzes the eye-related data and the target-related data and determines a relation between a change in the visual axes data and a change of said distance to the target during movement, enabling to extract data indicative of NPC and/or CRP from said relation. Also system and method are presented for performing a cover test to detect eye misalignment of a patient.

### CROSS-REFERENCE TO RELATED APPLICATIONS

This application is a continuation-in-part of International Application No. PCT/IL2025/050673 filed on Aug. 7, 2025, which claims the priority benefit under 35 U.S.C. § 119 of U.S. Provisional Patent Application No. 63/680,409, filed on Aug. 7, 2024, the contents of each are hereby incorporated in its entirety by reference.

### TECHNOLOGICAL FIELD AND BACKGROUND

The present disclosure relates generally to systems and methods of diagnosing eye disorders.

Near Point of Convergence/Convergence Recovery Point (NPC/CRP) measurements of a patient are very common and today are performed with special ruler, subjective responses of the patient, and practitioners unassisted (without any device) observations of the patient's eyes behavior.

Cover testing is the gold standard for the objective method for determining the presence, type, and magnitude of ocular misalignment (strabismus).

Performing the full cover tests takes time, requires high skills from the practitioner, and is not accurate, because of manual methods used.

More accurate measurements of deviation eye angles are required to monitor disease progress and for decisions for treatment that can be surgery for severe cases, special prisms, and vision therapy.

### GENERAL DESCRIPTION

Convergence insufficiency (CI) is characterized by a decreased ability to converge the eyes and maintain binocular vision while focusing on a near target. NPC/CRP measurements are a golden standard today for CI detection because of the test's simplicity and providing the results in the form of a simple number that may be recorded and compared with the results of future and past measurements. Furthermore, NPC/CRP measurements serve as indicator (i.e. biomarker) to a large set of other pathologic problems, like intermitted exophoria, vertical phoria, etc.

Current procedures of NPC/CRP measurement are performed manually by a specialist (e.g., ophthalmologist or optometrist) using a special ruler with a fixation target installed on it. The patient fixates on the target, and by bringing the target closer to the patient's eyes, the distance from the patient's eyes at which fixation is lost (i.e., the patient sees double) is measured. This distance/point is called the Near Point of Convergence (NPC) and is denoted as d_{NPC}. The specialist then gradually moves the target away from patient's eyes and at some stage the patient recovers his vergence and indicates to the specialist that now he sees single. This distance/point is called Convergence Recovery Point (CRP) and is denoted d_{CRP}. Typically, d_{NPC}≠d_{CRP}.

There exist conflicting findings for normative values of NPC/CRP measurements, originating, among other reasons, from inaccuracies and insufficient repeatability of the manual measurement technique.

One of the aspects of the present disclosure relates to improvement of NPC/CRP measurements for diagnostics of CI and some other eye misalignment syndromes, by providing a novel semi-automatic and objective NPC/CRP measurements with the help of an eye tracking system capable of monitoring a change in data indicative of the eyes' visual axes in space and/or a change of a distance between the limbuses of the eyes with high accuracy during a measurement session (during which a distance from the eyes to a target on which the eyes are required to be focused continuously changes). In this connection, it should be understood that the currently used fully manual procedures are based on a mixture of subjective (patient verbal response based) and objective (direct unassisted observation by a specialist) data.

The present disclosure uses computer vision to measure both the target location and data indicative of the fixation of the patient on the target during an NPC/CRP measurement. The measurement of fixation of the patient's eyes on the target requires measurement of their visual axes' locations in space.

The present disclosure provides a novel eye tracking technology utilizing determination of data indicative of visual axes of the eyes, associated with eyes' vergence, in a measurement session during which a distance from the eyes to a target, onto which the eyes are required to be focused, is being changed (typically, by moving the target). Such determination of the data indicative of the visual axes may include measurements of the location of the visual axis of each eye in space, enabling to directly deduce the convergence/divergence of the visual axes. Additionally, or alternatively, the determination of the data indicative of the visual axes includes accurate measurement of a distance between limbus centers of the eyes. Since it is known that such distance decreases as a person focuses on a near target, reduced or delayed convergence of the eyes is indicative of that the eyes do not rotate inward normally during near focusing tasks, resulting in cessation of decrease in limbus-to-limbus distance.

It should be noted that the term “eyes'vergence” used in the present disclosure refers to the simultaneous (coordinated) movement of both eyes (eyes' optical/visual axes) in opposite directions. “Eyes'convergence” refers specifically to the inward turning of the eyes when trying to fixate at near objects, whereas “eyes'divergence” refers to the outward turning of the eyes when trying to focus on far objects.

Thus, the technique of the present disclosure enhances current manual procedures with objective data about the patient's lost fixation on the target (NPC data d_{NPC}), and when the patient's eyes regain it back (CRP data d_{CRP}.). The technique of the present disclosure not only provides improved results as compared to the known manual procedure, but in addition allows to store the whole eye movements data that may be used for better diagnostics and monitoring of improvement or deterioration of patient's conditions.

It should be noted that the present disclosure may utilize any known eye tracking technology able to provide the measured eye-related data indicative of visual axes of the eyes, associated with eyes' vergence. Possible eye tracking technologies include camera/image-based technologies, electro-oculography (EOG), eye-attached tracking, optical tracking, and video-based combination of pupil/corneal reflection. The optical tracking may include photo-oculography (POG) and video-oculography (VOG) methods utilizing reflected light source (typically infrared light) from the eye and sensed by a video camera or some other optical sensor. The video-based combined pupil/corneal reflection method provides two key features, i.e., the corneal reflection (known as the Purkinje reflections) and the pupil center to monitor the regard point. This method can be used with low-priced cameras and is able to compute data indicative of the visual axes in real time.

Additionally, it should be noted that the present disclosure may utilize any known target tracking technology to provide the measured target-related data. Such technology includes camera/image-based technologies, infrared (IR) time-of-flight (ToF) or Lidar, ultrasonic sensors, inertial measurement unit (IMU) on target, and RFID with field strength measurement. For the sake of simplicity, the principles of the present disclosure will be described below using the camera/image-based technology.

Thus, according to one broad aspect of the present disclosure, it provides a system for measuring near point of convergence (NPC) and/or convergence recovery point (CRP) of patient's eyes, the system comprising:
an eye tracking device configured and operable to provide measured eye-related data of the eyes being indicative of visual axes of the eyes associated with eyes' vergence during a measurement session; a target tracking device, being calibrated with respect to the eye tracking device, and being configured and operable to monitor distances from the eyes to a moving target, onto which the eyes are required to be focused during said measurement session, and to provide measured target-related data indicative of a relative target location relative to the eyes; and
a memory and processing circuitry configured and operable to analyze the measured eye-related and the measured target-related data and determine a relation between a change in the data indicative of the visual axes and data indicative of a change of said distance to the target during movement of said target during the measurement session, and to extract data indicative of said NPC and/or CRP from said relation.

The memory and processing utility may be configured and operable to analyze said measured eye-related data of the eyes and determine the change in the data indicative of visual axes of the eyes in space during the measurement session.

In some embodiments, the data indicative of the visual axes comprises locations of the visual axes in space. In this case, the relation describes a mismatch between a point of intersection of said visual axes and the distance to a control location of the target corresponding to a condition of loss of focus of the eyes on said target.

In some embodiments, the data indicative of the visual axes associated with the eyes'vergence comprises a distance between limbus centers of the eyes. The relation describes a change of the distance between the lumbus centers as a function of the change of said distance to the target, enabling to identify a value of the distance between the limbus centers corresponding to a predetermined change in said function, said value corresponding to eyes' vergence condition of loss of focus of the eyes on said target.

The measurement system or the target tracking device may further comprise a target movement assembly comprising an elongated scale aligned with a rail on which the target is mounted for controlled movement along said rail, thereby enabling to monitor the distances from the subject's eyes to the target during said controlled movement of the target. A movement pattern of the target is calibrated with the eye-related data acquisitions pattern of the eye tracking device thereby enabling to determine said relation between the change in the data indicative of the visual axes associated with the eyes'vergence and the change of the distance to the target during said movement.

In some embodiments, the measured eye-related data is image data.

In some embodiments, the eye tracking device may comprise at least one imaging device, each configured and operable to perform imaging of both eyes of the patient during the measurement session, thereby providing image data of the eyes being the measured eye-related data.

In some embodiments, the eye tracking device comprises at least two imaging devices, associated with two eyes, respectively, such that at least one of said at least two imaging devices performs imaging of one of the two eyes and at least one other of the at last two imaging devices performs imaging of the other of the two eyes during the measurement session, thereby providing the measured eye-related data of the eyes.

In some embodiments, the eye tracking device comprises at least two pairs of imaging devices, associated with two eyes, respectively, wherein each pair of the imaging devices performs imaging of the respective eye during the measurement session, thereby providing the measured eye-related data of the eyes.

In some embodiments, the target tracking device comprises at least one imaging device each associated with the two eyes and configured and operable to perform imaging of the moving target and provide the measured target-related data, each of said at least one imaging device being located at calibrated distance from the one or more imaging devices of the eye tracking device.

In some embodiments, the target tracking device comprises at least two imaging devices comprising at least one imaging device associated with one of the two eyes and at least one other imaging device associated with the other of the two eyes, said at least two imaging devices being configured and operable to perform imaging of the moving target and provide said measured target-related data, said at least two imaging devices being located at calibrated distances from the one or more imaging devices of the eye tracking device.

According to another broad aspect of the present disclosure, it provides a method for measuring near point of convergence (NPC) and/or convergence recovery point (CRP) of patient's eyes, the method comprising:
providing measured eye-related data of the patient's eyes, indicative of visual axes of the eyes associated with eyes' vergence during a measurement session, and providing measured target-related data collected during the measurement session during which a target, onto which the eyes are required to be focused, is controllably moved with respect to the eyes, said measured target-related data being indicative of a relative target location relative to the eyes;
processing said measured eye-related data and said measured target-related data and determining a relation between a change in the data indicative of visual axes of the eyes and data indicative of a change of said distance to the target during movement of said target, and extracting data indicative of said NPC and/or CRP from said relation.

Said processing may comprise: analyzing the measured eye-related data and determining the change in said data indicative of the visual axes in space during the measurement session.

In some embodiments, said data indicative of the visual axes comprises locations of the visual axes in space, said relation describing a mismatch between a point of intersection of said visual axes and the distance to a control location of the target corresponding to a condition of loss of focus of the eyes on said target.

In some embodiments, said data indicative of the visual axes associated with the eyes'vergence comprises a distance between limbus centers of the eyes, said relation describing a change of said distance between the lumbus centers as a function of the change of said distance to the target, enabling to identify a value of the distance between the limbus centers corresponding to a predetermined change in said function, said value corresponding to eyes' vergence condition of loss of focus of the eyes on said target.

In some embodiments, said data indicative of the visual axes associated with the eyes'vergence comprises a distance between limbus centers of the eyes, said relation describing a change of said distance between the lumbus centers as a function of the change of said distance to the target comprises a decrease in said distance between the lumbus centers as the target is moved from a larger distance towards the eyes, data indicative of a stopped decrease in said distance between the lumbus centers as the target is moved from a larger distance towards the eyes is indicative of NPC.

In some embodiments, said data indicative of the visual axes associated with the eyes'vergence comprises a distance between limbus centers of the eyes, said relation describing a change of said distance between the lumbus centers as a function of the change of said distance to the target comprises an increase in said distance between the lumbus centers as the target is moved from a relatively close distance towards further away from the eyes, data indicative of a gradual increase in said distance between the lumbus centers as the target is moved towards further away from the eyes is indicative of CRP.

In some embodiments, said measured eye-related data is image data.

In some embodiments, said providing of the measured eye-related data comprises performing image acquisitions by at least one imaging device, each associated with two eyes, and providing said measured eye-related data.

In some embodiments, said providing of the measured eye-related data comprises performing image acquisitions by at least two imaging devices associated with two eyes, respectively, and providing said measured eye-related data.

In some embodiments, said providing of the measured target-related data comprises imaging the target while said target is moved along a rail aligned with an elongated scale, and monitoring the distances from the subject's eyes to the target during the movement of the target, a movement pattern of the target being calibrated with the measured eye-related data acquisitions pattern.

In some embodiments, said providing of the measured target-related data comprises imaging of the moving target by at least one imaging device, each associated with the two eyes, each of said at least one imaging device being located at calibrated distance from one or more imaging devices of the eye tracking device.

In some embodiments, said providing of the measured target-related data comprises imaging of the moving target by at least two imaging devices associated with the two eyes, respectively, said two imaging devices being located at calibrated distances from the respective two pairs of the imaging devices of the eye tracking device.

In some other aspects of the present disclosure, it provides improvement of cover test procedure measurements for diagnostics of eyes alignment disorders, by providing a novel semi-automatic and objective eyes deviation measurement with the help of an eye tracking system capable of measuring the visual axis of the eye with high accuracy. In this connection, it should be understood that the accuracy of currently used fully manual procedures is limited by available mechanical prisms and subjective observations by practitioner.

The main ophthalmological terms are used to describe alignment-related eye conditions:

Tropia/heterotropia describes strabismus, i.e., a constant, visible misalignment of the eyes where one eye deviates from the correct position even when both eyes are open and attempting to fixate on the same object. This misalignment is present under normal viewing conditions. The tropia is detected using “cover-uncover” test in which one eye of a patient is covered (with an occluder) while the patient fixates on a target. If the other, uncovered, eye moves to take up fixation, a tropia is present. The movement reveals which of the two eyes is deviating and in what direction. Types of tropia include esotropia (eye turns inward), exotropia (outward), hypertropia (upward), and hypotropia (downward). Treatment options of tropia include glasses, prisms, vision therapy, or surgery depending on severity.

Phoria/heterophoria describes a latent tendency for eye misalignment that is normally controlled by brain's fusional system. In phoria, the eyes remain aligned under normal binocular viewing conditions, and the misalignment becomes apparent only when fusion is disrupted (e.g., covering one eye). When the occluder is removed, the eyes realign through fusional mechanisms. The phoria is detected using an “alternate cover” test which detects both tropia and phoria (total deviation). During the test, the cover/occlude is alternated rapidly between eyes, preventing fusion, and reveals any latent deviation (phoria) in addition to manifest deviation (tropia). It, thus, shows the maximum deviation when fusion is completely disrupted. Types of phoria include esophoria (tendency to turn inward), exophoria (outward), hyperphoria (upward). As with tropia, treatment options include corrective lenses, prism glasses, or vision therapy.

Amblyopia, commonly called “lazy eye,” is a vision development disorder in which one eye fails to achieve normal visual acuity, even with corrective lenses. It occurs when the brain and the affected eye don't work together properly, and the brain begins to favor the stronger eye. It is the most common cause of vision impairment in children. Amblyopia can be tricky to detect because children often don't realize anything is wrong. Early detection is crucial because amblyopia is most treatable during childhood while the visual system is still developing. Treatment typically involves correcting any underlying refractive error with glasses and then forcing the brain to use the weaker eye through patching or eye drops that blur the stronger eye.

Cover testing is the gold standard for the objective method for determining the presence, type, and magnitude of ocular misalignment (strabismus). It is typically performed by the ophthalmologist, orthoptist, or ophthalmic technician. Cover testing can be done at both near and distance relative to the eye. When performed at near, an accommodative fixation target is held at a distance of 33-40 centimeters. At distance, the patient fixates on a target at a distance of 6 meters.

The inventors have realized that performing the cover testing at both near and distance is clinically essential for several reasons. First, many patients show different amounts or even different types of misalignments at near vs. distance, for example, a patient might have orthophoria (no deviation) at distance, but exophoria at near, or vice versa. The deviation magnitude often differs significantly between the two distances. Second, specific conditions require distance differentiation. For example, convergence insufficiency may show normal alignment at distance, but exophoria/exotropia at near. Such conditions, being very common and causing reading difficulties and eye strain, would be missed if testing is done only at distance. Another condition which may be missed if only near testing is performed, is divergence insufficiency where esophoria/esotropia is worse at distance than near. Performing the cover testing at both near and distance has also the following important treatment implications: if near deviation differs from distance, bifocals or progressive lenses may be needed, prism prescription may differ for near vs. distance, and vision therapy protocols may depend on where the problem manifests.

It should also be noted that despite being golden standard, the full cover test takes time, especially when exact numbers of eyes deviations are needed, and thus different prisms may be used in the test procedure in order to find the right deviation. The larger the deviation, the less accurate it becomes, because the step between available consecutive prisms increases with increase of deviation angle that needs to be compensated. The step starts from 2 prismatic diopters for small angles (less than 10 degrees deviation) and increases to 5 diopters for larger angles. It is also noted that very small, around 1 degrees eye deviation, is difficult to identify for the practitioner and requires high skills. Exact measurements of deviation angles is required to monitor disease progress and to decide about the treatment that can be surgery for severe cases, special prisms, and vision therapy.

The present disclosure provides a high accuracy eye tracking system to perform direct measurement of eyes'misalignments in dynamic conditions, thus, possibly, simplifying the procedures, and even eliminating the need for prism-based tests.

The present disclosure uses computer vision to measure both the target location and the movements of subjects' eyes, while the eyes are trying to fixate on the target. These measurements require measurement of patients' visual axes' dynamics in space. The inventors provide and use the eye tracking technology to measure the location of the visual axis of each eye in space and directly deduce the convergence/divergence and other dynamics of the visual axes.

The eye tracking technology suitable to be used in the technique of the present disclosure may be that described in U.S. Pat. No. 12,056,274, assigned to the assignee of the present application, as well as U.S. Pat. No. 12,066,624 where the present inventor is a co-inventor. These patents are incorporated herein by reference.

The technique of the present disclosure enhances current manual procedures with objective and accurate data about the patient's loss of fixation on the target by occluded eye, and when the patient's eye regains the fixation back. The technique of the present disclosure not only provides improved results as compared to the known manual procedure, but in addition allows storing the measured movement data of the eye that may be used for better diagnostics and monitoring of improvement or deterioration of patient's conditions.

Thus, according to a further broad aspect of the present disclosure, it provides a system for performing a cover test to detect eye misalignment of a patient, the system comprising:
a target assembly comprising a fixation target exposed to be visible to the patient, and an optical marker associated with the fixation target;
an eye occluder operable to selectively block and unblock vision of one of the left and right eyes of the patient, such that said one of the eyes sequentially experiences cover-uncover conditions during the cover test;
an eye tracking device configured and operable to perform first image acquisitions of each of left and right eyes of the patient and provide measured eye-related data of each of the left and right eyes, said measured eye-related data being indicative of a first time patterns of three-dimensional position and angular orientation of visual axes of the eyes during the cover test;
a target tracking unit being calibrated with respect to the eye tracking device, the target tracking unit being configured and operable to perform second image acquisitions of said optical marker during the cover test synchronously with the first image acquisitions performed by the eye tracking device, and generate data indicative of a second time pattern of a three-dimensional spatial location of the fixation target during the cover test;
a processing and memory circuitry configured and operable to carry out the following: receive and process said data indicative of the first time pattern and said data indicative of the second time pattern, and determine, for each pair of synchronously performed first and second image acquisitions, data indicative of angular deviation of the visual axis of each of the left and right eyes relative to a theoretical fixation line extending from the respective eye to said fixation target, thereby obtaining data indicative of temporal dynamics of said angular deviations during the cover test; and generate output data indicative thereof.

The optical marker may comprise a fiducial pattern enabling six-degree-of-freedom pose estimation.

In some embodiments, the system is configured and operable to measure said angular deviations of the visual axis of each of the eyes relative to said theoretical fixation line at a target distance from the respective eye in a range from 33 cm to 6 meters.

The data indicative of temporal dynamics may comprise saccadic velocity and acceleration profiles of the eyes during cover-uncover transitions of the eye condition.

The processing and memory circuitry may be configured and operable to apply neural network processing to said first and second time patterns to identify and exclude erroneous eye tracking data caused by blinks or artifacts.

According to yet further broad aspect of the cover test relating invention, it provides a method for monitoring eye misalignment of a patient, the method comprising:
providing measured eye-related data of patient's eyes, indicative of visual axes of the patient's eyes relative to optical axis of the patient's eyes;
providing first and second measured data, synchronously recorded during a selected cover test session on the patients' eyes, comprising first image acquisitions of each of left and right eyes of the patient while fixating on a fixation target for at least one distance of the fixation target from the eye, and second image acquisitions of an optical marker associated with the fixation target;
processing said first and second measured data and determining data indicative of respective first time pattern of three-dimensional position and angular orientation of visual axes of the eyes and second time pattern of a three-dimensional spatial location of the fixation target; and
utilizing the measured eye-related data of patient's eyes and processing the first and second time patterns and determining, for each pair of synchronously performed first and second image acquisitions, data indicative of angular deviation of the visual axis of each of the left and right eyes relative to a theoretical fixation line extending from the respective eye to the fixation target, said angular deviation of the visual axis of each of the left and right eyes being indicative of temporal dynamics of the angular deviations during the cover test session, enabling detection of eye misalignment.

The selected cover test may include at least one of the following: cover/uncover test and alternate cover test.

The providing of the first and second measured data may include accessing/receiving such measured data from a storage device. Alternatively, providing the first and second measured data comprises performing the cover test session by operating a system comprising:
a target assembly comprising a fixation target exposed to be visible to the patient, and an optical marker associated with the fixation target;
an eye occluder operable to selectively block and unblock vision of one of the left and right eyes of the patient, such that said one of the eyes sequentially experiences cover-uncover conditions during the cover test;
an eye tracking device configured and operable to perform the first image acquisitions of each of left and right eyes of the patient and provide the first measured data of each of the left and right eyes, indicative of the first time pattern of three-dimensional position and angular orientation of visual axes of the eyes during the cover test session;
a target tracking unit being calibrated with respect to the eye tracking device, the target tracking unit being configured and operable to perform the second image acquisitions of said optical marker during the cover test synchronously with the first image acquisitions and generate data indicative of the second time pattern of a three-dimensional spatial location of the fixation target during the cover test.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

In order to better understand the subject matter that is disclosed herein and to exemplify how it may be carried out in practice, embodiments will now be described, by way of non-limiting examples only, with reference to the accompanying drawings, in which:

FIG. 1 is a schematic illustration of the configuration and operation of the NPC/CRP measurement system of the present disclosure;

FIG. 2 shows an exemplary configuration of an NPC/CRP measurement system of the present disclosure including a total of 6 cameras, 3 cameras for each eye;

FIG. 3 exemplifies a method of the present disclosure for NPC/CRP measurements;

FIGS. 4A-4C show a few specific examples of NPC/CRP measurement techniques of the present disclosure, utilizing visual axis-based approach for NPC/CRP measurement and limbus distance based approach for NPC/CRP measurement, wherein FIG. 4A shows the measurement of NPC while the target is moved towards the eyes, FIG. 4B shows the measurement of CRP while the target is moved away from the eyes, and FIG. 4C shows the change in the distance between the centers of limbuses;

FIG. 5 exemplifies the measurement procedure using the measurement system configured according to the present disclosure;

FIGS. 6A-6B exemplify measurement results of the technique of the present disclosure, showing a condition of the abnormal change in the convergence/divergence of the visual axes of the patient's eyes determined via a change of the distance between the centers of the limbuses of the eyes;

FIG. 7 is a general illustration of the cover/uncover test procedure;

FIGS. 8A and 8B exemplify the cover/uncover tests to identify heterotropia/heterophoria condition (FIG. 8A) and the probability of amblyopia condition (FIG. 8B);

FIG. 9 is a general illustration of the alternate cover test procedure combined with prism measurement;

FIGS. 10A and 10B exemplify the alternating cover test (FIG. 10A) combined with prism measurement (FIG. 10B);

FIG. 11 shows a cover test procedure measurement system according to the principles of the present disclosure;

FIG. 12 is a flow diagram exemplifying a method of the present disclosure for detecting eye misalignment of a patient; and

FIGS. 13A to 13C show an exemplary cover test procedure using the measurement system of the present disclosure.

### DETAILED DESCRIPTION OF EMBODIMENTS

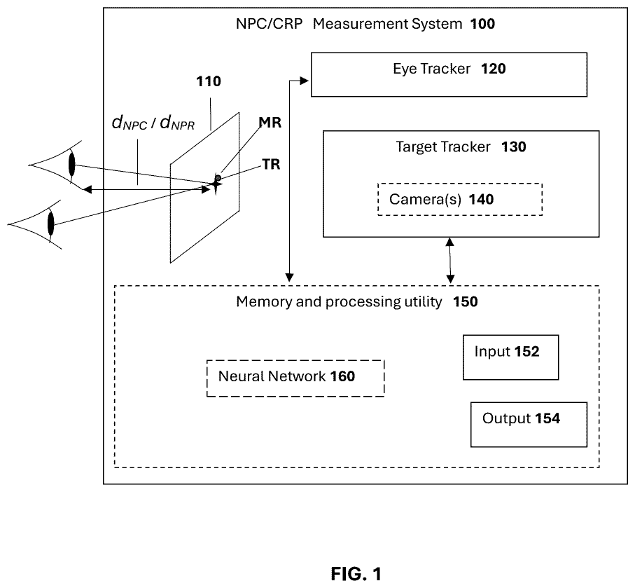

FIG. 1 shows schematically the NPC/CRP measurement system 100 according to the principles of the present disclosure. The system 100 includes an eye tracking device 120, a target tracking device 130. The location and orientation of target tracking device 130 is calibrated with respect to the location and orientation of eye tracker device 120.

As will be described below, with such calibration when eye tracking device finds orientation and location of eyes (visual axis, optical axis, limbus center), it is possible to calculate relative location of the eyes with respect to a target onto which the eyes are required to be focused during the target movement, and consequently it is possible to calculate the distance of eye features being tracked, such as limbus, from the target. Additionally, the calibration allows to calculate if visual axes are pointing towards the target and other relations between the target TR and the eyes.

The system 100 is associated (i.e., includes or used with) a target assembly 110 which is configured to allow controllable change of a distance between the target (on which patient's eyes are focused) and the patient's eyes during a measurement session. Typically, this can be implemented by movement of the target. Also, the system 100 is associated with (i.e., includes or is connectable to) a memory and processing circuitry 150.

The NPC is the last location (the smallest measured distance) of the target during the target's movement towards the eyes, at which both visual axes coincide with the location of the target, and CRP is the first location (the closest to the eyes) of the target during the target's movement away from the eyes, at which target location is again coinciding with the convergence point of both visual axes.

The target assembly 110 may generally be of any known suitable configuration and typically may include a moving target TR and a ruler. In some embodiments, the target assembly 110 may utilize, for example, a modified Accommodation Convergence Rule (Bernell) ruler (not shown) and a target TR. The target may be equipped with a special marker MR. The target TR is used to fixate the patient's eyes on.

The eye tracker device 120 is configured and operable to provide measured eye-related data of the eyes being indicative of visual axes of the eyes in space during the measurement session. The eye tracker device 120 may generally be of any known suitable configuration. The eye tracker device 120 includes at least one camera associated with each eye of the patient and is configured and operable to measure the position and orientation of the optical axis of each eye of the patient. For example, the eye tracker device 120 may include an adjustable head-mounted apparatus with camera(s) in front of the participant's eyes.

In some embodiments, the eye tracker device 120 may be configured and operable to measure the position and orientation of the optical axis of each eye of the patient with no required calibration. Such eye tracker device 120 may be configured as described in U.S. Pat. Nos. 12,056,274 and 12,066,624, the entire disclosure of which, except for any definitions, disclaimers, and inconsistencies, is incorporated herein by reference.

It is known that the optical axis, being the symmetrical axis of the eye, is displaced from the visual axis which connects the target with the fovea. For the purposes of measuring NPC/CRP, data indicative of “behavior” of the visual axis is required. In some embodiments, the NPC/CRP is extracted from measurements of abnormal change in the convergence/divergence of the visual axes of the patient's eyes.

Normally, according to the given tasks, both visual axes of the patient's eyes are pointing to the target, i.e. when target moves closer, the eyes gradually converge so that their visual axes continue pointing at the target. When target moves further away, the eyes gradually diverge. When target is too close, at least one of the eyes becomes incapable of pointing to the target. This may be accompanied by a number of abnormal eye movements, like quickly switching between a state in which one of the eyes points to the target and the other eye does not, or one of the eyes starts slowly drifting away from the target.

To measure visual axes'locations in space and to determine whether the visual axis points towards the target TR, a one-time calibration step may be performed to determine the constant (per patient) displacement of the visual axis from the optical axis. Thus, the continuous tracking of the optical axes of the eyes provided by the eye tracker device 120 may be easily transformed into respective tracking of the visual axis, enabling measurement of the visual axes' locations in space.

In some embodiments (which may be alternative or additional embodiments), the visual axes behavior (data indicative of the visual axes of the eyes) can be determined from a measured distance between limbus centers of the eyes (i.e., points of intersection of the optical axes with the limbus plane) provided by the eye tracker device 120. The distance between limbus centers of the eyes changes as a function of the change of the measured distance to the target. When the target is moving towards the eyes in order to determine NPC, the distance between limbus centers gradually decreases, as the eyes continuously verge towards the target, i.e. while visual axis of each eye points towards the target. In this embodiment, the time when the distance between the centers of the limbuses stops decreasing, corresponds to the time when the patient fails to converge towards the target, and the distance from the target to the eyes at that moment is defined as NPC. When the target is moving away from the eyes, the distance between limbus centers gradually increases, while visual axis of each eye points towards the target. In order to determine CRP, the target initially is brought to the distance smaller than NPC and is gradually moved away from the eyes. At some distance, usually larger than NPC, the patient succeeds in pointing both of their visual axes towards the target. Starting from this point the angle between visual axes starts decreasing gradually and the distance between the centers of the limbuses starts gradually to increase. This point is defined as CRP. In these embodiments, the calibration of displacement of the visual axis from the optical axis may not be required.

The target TR position during the measurement session is tracked by the target tracker 130 which may include at least one imager (camera) 140. Such a camera may, for example, be a forward-facing camera mounted on the head-mounted apparatus of the eye tracker device 120.

As noted above, the target may be equipped with the marker MR which may be attached to the target TR in such a way that it does not block the target TR itself and that forward-facing camera will always have MR in its field of view, even when the target is very close to the eyes. During the measurement session, the distance between the patient's eyes and the target is being continuously changed while the patient's eyes follow the target with their gaze. The distance relation between the target TR and the MR is fixed, therefore the distance between the target TR and the eyes can be determined even if the camera of the target tracking device cannot properly image the target, which is typically smaller than the marker and is brought closer to the eyes, since the marker always can be imaged.

The target TR may be a small object, like a dot or a small letter. The eye tracking 120 is configured and operable to measure the relative eyes'location in 3D and is not exposed to the target and thus the measured eye-related data provided by the eye tracker 120 is not indicative of the relative target TR location.

The measured eye-related data of the eyes (which is indicative of the visual axes of the eyes) continuously provided by the eye tracker device 120 during a measurement session, and the measured target-related data indicative of the relative target location provided by the target tracking device 130 during said measurement session, enable to determine a relation between a change in said data indicative of the visual axes and a change of the distance to the target during movement of said target. This relation is indicative of the NPC and/or CRP

The memory and processing circuitry 150 is configured and operable to determine the relative location of the eye tracking device 120 and the target tracking device 130, as will be described below.

According to the present disclosure, the memory and processing circuitry 150 is capable of automatically and objectively analyzing and processing the measured eye-related data (provided by the eye tracking device) and measured target-related data (provided by the target tracking device) and determining at what distance the patient's eyes fail to verge (NPC) and when their ability to verge is restored (CRP). More specifically, the memory and processing circuitry 150 receives said measured eye-related data of the eyes from the eye tracking device 120 and receives said measured target-related data from the target tracking device 130. The received data is analyzed by memory and processing circuitry 150 to determine, from the measured eye-related data, the data indicative of visual axes of the eyes in space associated with eyes' vergence during a measurement session, and to utilize the visual axis related data together with the measured target-related data indicative of the relative target location relative to the eyes during the measurement session to determine the relation between the change in visual axes relating data and the change of said distance to the target during the measurement session. Then the memory and processing circuitry 150 extracts data indicative of said NPC and/or CRP from said relation.

In some embodiments, the measurement session and data analysis include a continuous measurement of the position and orientation of the visual axis of each eye (by the eye tracker 120) and a continuous measurement of the relative target location (by the target tracker 130). As noted above, the NPC is the last location (the smallest measured distance) of the target during the target's movement towards the eyes, at which both visual axes coincide with the location of the target, and CRP is the first location (the closest to the eyes) of the target during the target's movement away from the eyes, at which target location is again coinciding with the convergence point of both visual axes. The automatic and objective measurements with the eye tracking device and the target tracking device according to the present disclosure make the tests much more accurate and repeatable, as compared to the currently practiced manual tests. It should be understood that irrespective of whether the target movement is automatic or not) the measurement technique is automatic as compared to the conventional technique being manual in the meaning that such automatic technique does not rely/is independent of patient's response about his/her arrival to double-seeing the target as utilized in the manual technique.

Additionally, the measurement technique of the present disclosure provides recording of the dynamics of eyes' vergence loss/recovery, thus allowing to store important dynamic data, beyond the single number for NPC/NPR distance of a particular procedure.

In some embodiments, said data indicative of the visual axes comprises locations of the visual axes in space. The relation between the change in visual axes relating data and the change of said distance to the target during the measurement session describes a mismatch between a point of intersection of said visual axes and the distance to a control location of the target corresponding to a condition of loss of focus of the eyes on said target.

In some embodiments, the memory and processing circuitry 150 determines the data indicative of the visual axes by extracting, from the measured eye-related data provided by the eye tracker, a distance between limbus centers of the eyes. In this case, the relation between the change in the visual axes related data and a change of the distance to target related data describes a rate of change of the distance between the lumbus centers as a function of the change of the distance to the target. This enables to identify a value of the distance between the limbus centers corresponding to a predetermined change in said function, said value corresponding to eyes' vergence condition of loss of focus of the eyes on said target.

Turning back to FIG. 1, the memory and processing circuitry 150 is typically a computerized/electronic utility having data input and output 152, 154. The memory and processing circuitry is connectable (via wires or wireless communication of any known suitable type) to the eye tracking device 120 and the target tracking device 130. The memory and processing circuitry 150 receives the measured target-related data provided by the target tracking device 130 being indicative of relative locations of the target during the movement, and the measured eye-related data provided by the eye tracking device 120 being indicative of the relative locations of the eyes' optical/visual axes.

As noted above, the memory and processing circuitry analyses the measured eye-related data to determine the behavior of the eyes' visual axes in space by directly determining a change of the distance between limbus centers of the eyes from the images being acquired, and/or directly determining a change of the position and orientation of the optical axes of the eyes and transforming this into those of the visual axes of the eyes. The memory and processing circuitry 150 utilizes a pre-stored relative location (calibration data) of the eye tracker 120 and the target tracker 130 to determine the location of the eyes and the target at any given moment of time when receiving data from both the target tracker 130 and the eye tracker 120.

As noted above, the relative location of the eye tracker 120 and the target tracker 130 may be factory pre-calibrated. Specifically, in the embodiments where the eye tracking and the target tracking devices utilize cameras, this calibration may be performed by showing the eye tracker and target tracker cameras special markers with known relative location, thus enabling the calculation of the relative location of the eye tracker's and the target tracker's cameras by performing any known type of stereo cameras calibration procedure. Thus, the first and second measured target-related data can be processed by the memory and processing circuitry 150 to determine the NPC and/or CRP.

It should be noted that the measured eye-related data may be indicative of the erroneous behavior of patients' eyes, which can be properly identified by the memory and processing circuitry to disregard the respective measured eye-related data. To this end, a neural network 160 can be used, which is properly learned and trained to identify such erroneous data.

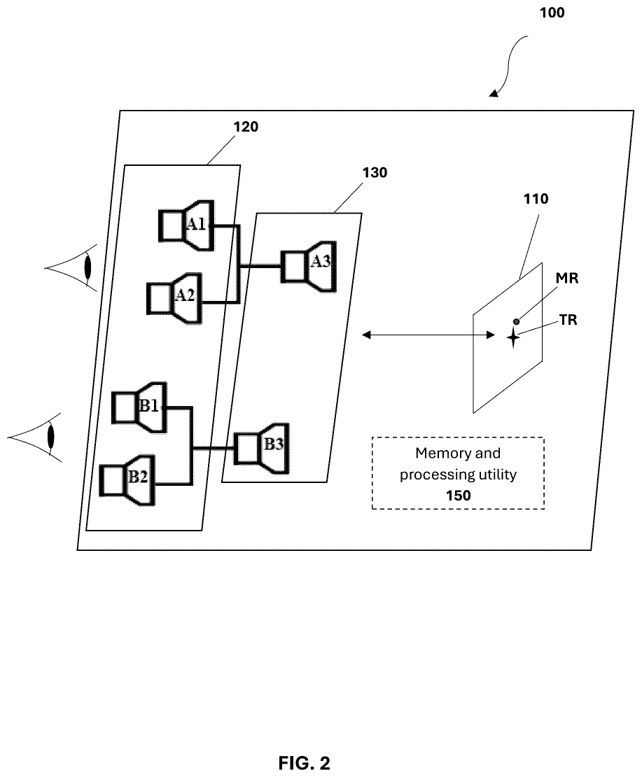

Reference is made to FIG. 2 exemplifying a configuration of a NPC/CRP measurement system 100 of the present disclosure including cameras/sensors of the eye tracking device 120 and target tracking de4vice 130 and in data communication with the memory and processing circuitry 150 described above. Generally, the eye tracking device 120 may include a single camera imaging both eyes of the patient; or may include one or more cameras for imaging each of the patient's eyes.

In the present non-limiting example of FIG. 2, the system 100 includes 6 cameras, including 3 cameras for each eye. In this example, the eye tracking device 120 includes at least two pairs of imaging devices (A1, A2) and (B1,B2), associated with two eyes, respectively, i.e., each pair of the imaging devices performs imaging of the respective eye during the measurement session, both pairs thereby providing the measured eye-related data of the eyes indicative of the visual axes of the eyes. The target tracking device 130 includes two imaging devices, A3 and B3, associated with the two eyes, respectively, and configured and operable to perform imaging of the moving target and provide measured target-related data about relative target location relative to the eyes during the measurement session. The two imaging devices, A3 and B3, of the target tracking device 130 are located at calibrated distances from the respective two pairs of the imaging devices, (A1, A2) and (B1,B2), of the eye tracking device. The measured eye-related data from the eye tracking device and the distance related measured target-related data from the target tracking device are used to determine the relation between a change in the data indicative of the visual axes and a change of the distance to the target during movement of the target being indicative of the NPC and/or CRP.

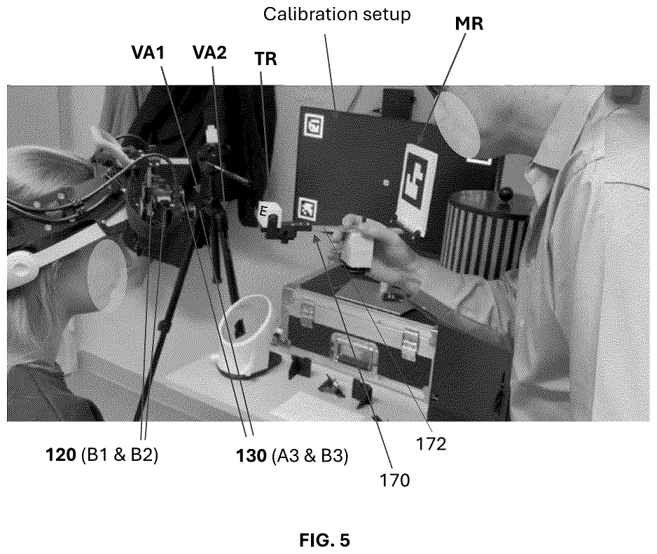

In some embodiments, the system 100 (or target tracking device 130) includes a target movement assembly (170 in FIG. 5) including an elongated scale aligned with a rail (172 in FIG. 5) on which the target TR is mounted for controlled movement along the rail, thereby enabling to monitor the distances from the patient's eyes to the target during the controlled movement of the target. A movement pattern of the target TR may be calibrated with the measured eye-related data acquisitions pattern of the eye tracking device 120 thereby enabling to determine the relation between the change in the data about the visual axes and the change of the distance to the target during the movement. Generally, either using automatic movement of the target or not, the distance measurements by the target tracking device are correlated with the eye-related data acquisition by the eye tracking device.

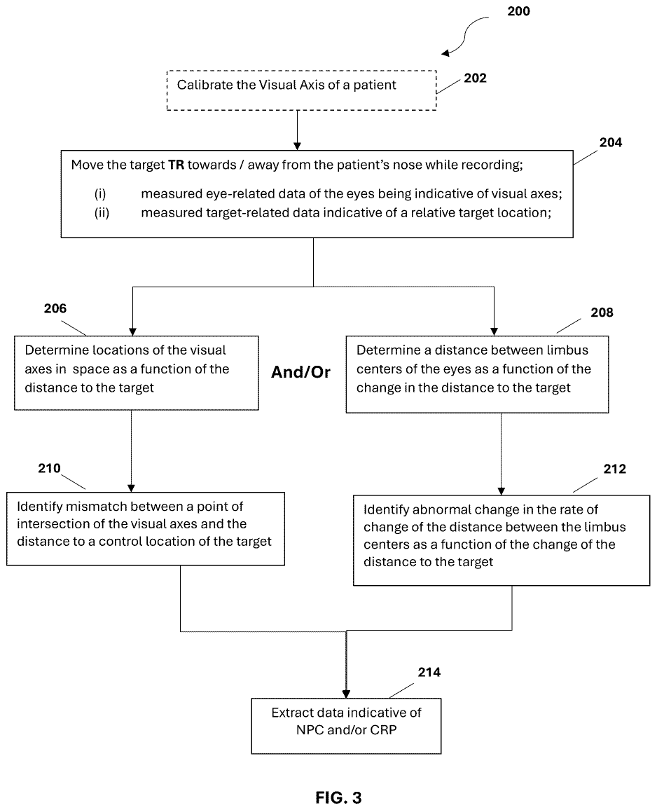

Reference is made to FIG. 3 showing a flow diagram 200 exemplifying a method of measuring near point of convergence (NPC) and/or convergence recovery point (CRP) of patient's eyes according to the principles of the present disclosure. In this non-limiting example, the eye tracking and the target tracking are performed using camera-based technology.

In the embodiments where the measured eye-related data is analyzed to extract locations of the visual axes in space, the method 200 is preceded by step 202 in which a calibration of the visual axis of the patient is performed. The specialist/operator inputs or accesses patient's ID and other patient-related data such as interpupillary distance (IPD), age, sex, special notes. Then, the head mounted device of the eye tracker 120 is put on the patient's head and the alignment procedure is performed. During the alignment procedure, the specialist/operator adjusts the position of the eye tracking camera(s) to the patient's IPD and head, so that the camera(s) is/are located in the correct position before the patient's eyes. The patient is guided to help fine-tune alignment to the eye tracking channels. In this step, the patient receives corresponding instructions, and the calibration is performed.

The calibration procedure may be performed as follows: The right eye's vision is blocked by a certain instrument. For 20-30 seconds, the patient's left eye is presented with a calibration target on a computer monitor. The patient follows the calibration target with their left eye. The eye tracking system 120 records these eye movements and calculates the optical axis location for each position of the calibration target, as well as the visual to optical axis angle for the left eye. In addition, it validates the visual axis tracking error as a sanity test to validate that the “Neural Network” works with the required accuracy on this specific patient. On top of it, this data may be used to improve eye tracking performance for the specific patient by annotating and adding the data to the training set. The same procedure is then repeated for the right eye.

In step 204 the measurement session is performed during which the target TR is moved (automatically or manually) towards (to determine NPC) or away from (to determine CRP) the patient's nose while the eye tracking device and target tracking device operate to provide and record (i) measured eye-related data of the eyes being indicative of visual axes of the eyes, and (ii) measured target-related data indicative of a relative target location.

Then, either step 206 or step 208, or both of them, is/are performed.

In step 206, the measured eye-related data and the distance measured target-related data are processed to determine locations of the visual axes in space as a function of the distance to the target (constituting the relation between a change in the visual axes related data and a change of distance to the target). This is followed by step 210 in which a mismatch between a point of intersection of the visual axes and the distance to a control location of the target is identified.

In step 208, the measured eye-related data and the distance measured target-related data are processed to determine a change in distance between limbus centers of the eyes (constituting the data indicative of the visual axes associated with the eyes'vergence) as a function of the change in the distance to the target. The rate of change of the distance between the centers of the limbuses corresponds to the rate of target movement towards or away from the eyes. This is followed by identification of the abnormal change in the rate of change of the distance between the limbus centers as a function of the change of the distance to the target (constituting the relation between a change in the visual axes related data and a change of distance to the target)—step 212. More specifically, a value of the distance between the limbus centers corresponding to a predetermined change in said function is identified, where this value corresponds to eyes' vergence condition of loss of focus of the eyes on said target. The point where an abnormal change in the distance between the centers of the limbuses is identified corresponds to NPC, when the target is moving towards the eyes (step 214). The point where the change between the centers of limbuses starts to correspond to the rate of the target movement away from the eyes is identified as CRP (step 214).

The operation of the system 100 may be properly verified. To this end, manual measurements by the specialist/operator are performed simultaneously with the measurements obtained by the eye tracking system 120. The target TR is located directly in front of the patient's eyes to be focused on and is moved slowly towards the patient's nose at a slow rate until the patient sees double (diplopia) (NPC). At this NPC break point, the patient tells the specialist/operator that he/she sees double and also presses a button on the keyboard at the same time. The target is then backed away from the patient's nose at the same rate until the patient's eyes see a single, indicating the CRP. The patient then tells the specialist/operator that the eyes see single again while simultaneously pressing the same button. This step is repeated (the “NPC/CRP test”) several times (at least 3 times) for each patient. During this step, the eye tracking system 120 also records the video of the eyes and of the target.

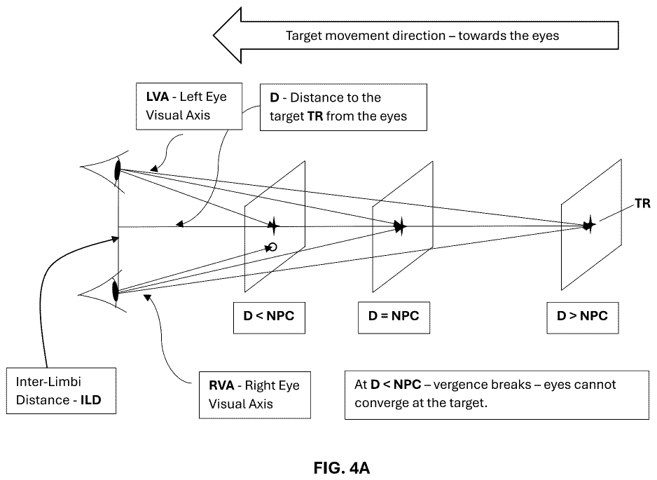

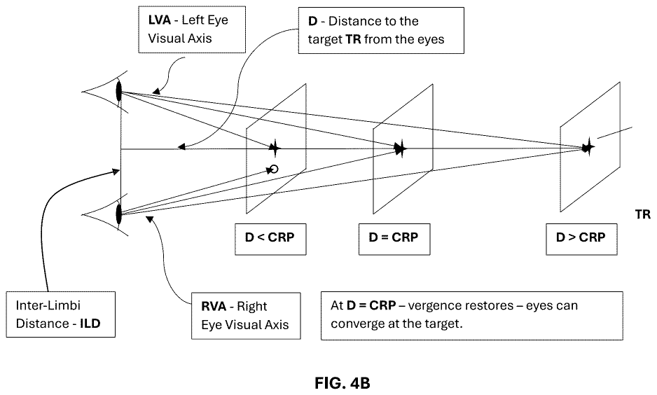

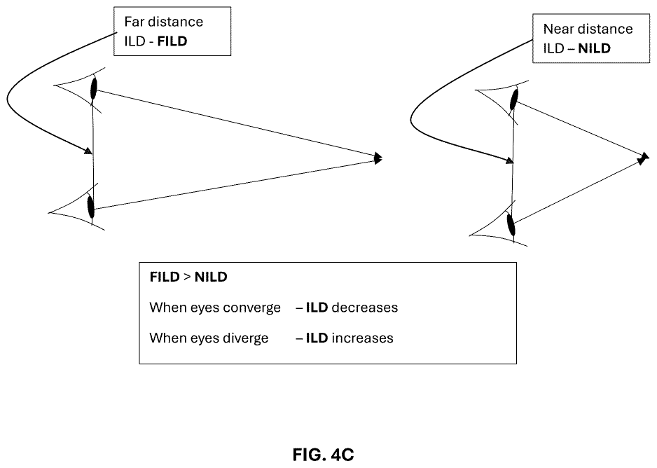

Reference is made to FIGS. 4A-4C exemplifying NPC/CRP measurement techniques of the present disclosure, utilizing visual axis-based approach for NPC/CRP measurement and limbus distance-based approach for NPC/CRP measurement.

FIG. 4A shows NPC measurement. The target TR starts at the far distance D and is gradually moved towards the eyes, while the patient is instructed to focus his gaze on the target all the time. The visual axis of left eye LVA, the visual axis of the right eye RVA, the distance between centers of limbuses of both eyes (Inter-Limbi distance—ILD), the distance D of the target TR to the eyes, as well as location of the target TR in space are measured. While the target moves closer, and the patient maintains the gaze of both of his eyes on the target, the angle between LVA and RVA increases and ILB decreases (FIG. 4C). The Near Point of Convergence, NPC, is defined as a minimal distance from the target TR to the eyes at which vergence breaks, i.e. the patient cannot verge both of his eyes towards the target. At this point the patient starts seeing double or suppresses images from one of the eyes. Objectively, this is characterized by the fact that at least one of the visual axes stops pointing to the target. Also, it is characterized by the fact that the angle between LVA and RVA stops increasing and the distance ILB stops decreasing. The distance ILB can even start slightly increasing, when the patient suppresses an image from one of his eyes, and the suppressed eye goes to the more natural position (e.g., directed straight).

Thus, the data indicative of the visual axes associated with the eyes'vergence includes a distance between limbus centers of the eyes. The relation describing a change of said distance between the lumbus centers as a function of the change of said distance to the target includes a decrease in said distance between the lumbus centers as the target is moved from a larger distance towards the eyes, data indicative of a stopped decrease in said distance between the lumbus centers as the target is moved from a larger distance towards the eyes is indicative of NPC

FIG. 4B shows CRP measurement. The target TR starts at the close distance D to the eyes, closer than NPC, and gradually moves father away from the eyes to the distance much larger than NPC. As with NPC, the visual axis of left eye (LVA), the visual axis of the right eye (RVA), the distance between centers of limbuses of both eyes ILD, the distance D of the target TR to the eyes, as well as location of the target TR in space are measured. While target TR moves further away, the patient attempts to verge both of his eyes on the target. Usually, he fails to do it on the distances that are smaller than NPC (FIG. 4A). Convergence Recovery Point, CRP, is defined as a minimal distance at which the patient successfully converges at the target. At this point, subjectively, the patient starts seeing the target TR as single again (if he saw it as double before). Objectively, both of his visual axes (LVA and RVA) start pointing to the target. Starting from this point, while target TR continues to move further away, distance D between the centers of the limbuses starts gradually increasing (FIG. 4C) and the angle between the visual axes starts gradually decreasing, corresponding to the distance D from the target TR to the eyes.

Thus, the data indicative of the visual axes associated with the eyes'vergence includes a distance between limbus centers of the eyes. The relation describing a change of said distance between the lumbus centers as a function of the change of said distance to the target includes an increase in said distance between the lumbus centers as the target is moved from a relatively close distance towards further away from the eyes, data indicative of a gradual increase in said distance between the lumbus centers as the target is moved towards further away from the eyes is indicative of CRP.

FIG. 5 exemplifies the measurement procedure performed using the measurement system configured according to the present disclosure. As shown, the system includes the eye tracking device 120 and the target tracking device 130 and further includes/utilizes the target movement assembly 170. The latter includes an elongated scale (not shown) aligned with the rail 172 on which the target TR is mounted for controlled movement along said rail (movement path), and also includes a marker MR where a change in the location of the marker with respect to the target during the measurement session is known. In this specific example the target is in the form of a small letter “E” and the marker is in the form of a duck. In this specific condition of the measurement session the target is much closer to the patient's eyes than the target. In this non-limiting example, the target tracking device 130 includes two forward-facing cameras A3 and B3 associated with the two eyes of the patient, i.e., each configured for monitoring a change of the distance between the respective eye and the target TR. It should be noted that the controlled movement of the target may be manual or automatic or semi-automatic. The eye tracking device 120 includes a pair of cameras associated with/imaging each eye—one pair of such cameras B1 and B2 being seen in the figure. As shown the forward-facing cameras of the target tracking device are mounted on the head-mounted apparatus of the eye tracking device 120. Also shown in the figure is a calibration setup which may be used once for determining a calibrated angular orientation between the optical and visual axes for each eye. Also shown are the two visual axes, VA1 and VA2, of the two eyes. The visual axes are drawn on the figure after calculation (by the memory and processing circuitry 150), to demonstrate the convergence of both visual axes of the patient on the target TR.

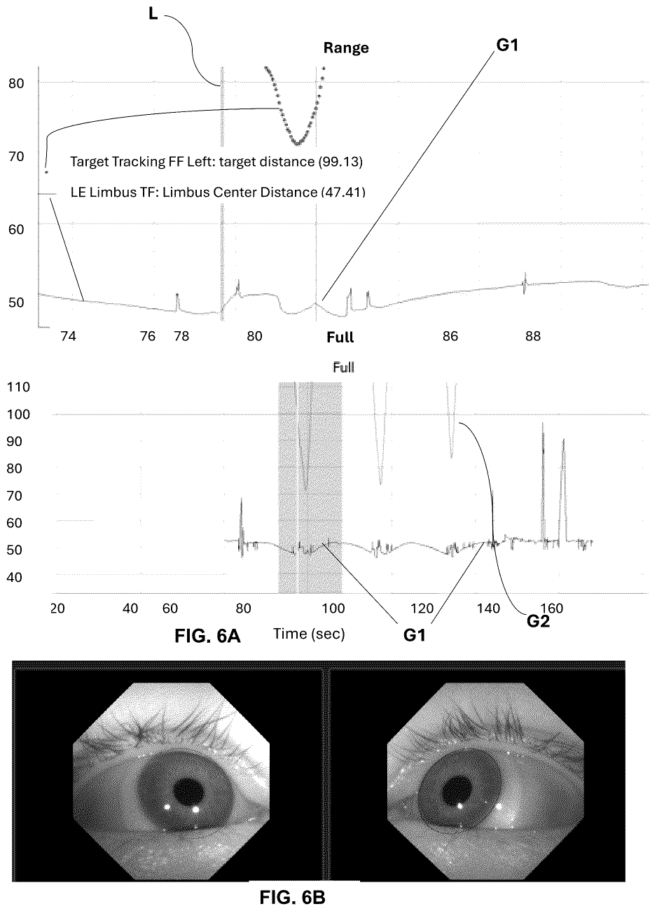

Reference is made to FIGS. 6A and 6B exemplify measurement results of the technique of the present disclosure, showing a condition of the abnormal change in the convergence/divergence of the visual axes of the patient's eyes determined via a change of the distance between the centers of the limbuses of the eyes.

FIGS. 6A-6B exemplify the following test. Practitioner moves the target TR bringing it gradually closer towards the patient's eyes till the target TR reaches some distance that is smaller than NPC (as exemplified in FIG. 5). At this point, the practitioner stops moving the target and gradually starts moving it in the opposite direction further away from the eyes and stops on some distance from the eyes that is larger than RCP. Then, the practitioner repeats this sequence 2 more times. During this test the patient tries to maintain the binocular fixation on the target TR all the time. When the distance is smaller than NPC, the patient fails to maintain the binocular fixation on the target TR, and when the distance is larger than RCP the patient regains binocular fixation.

In this specific example, the forward-facing cameras of the target tracking device are tracking the marker MR that is physically connected to the target TR via a (aluminum) ruler. The relative location of the target TR and marker MR is factory calibrated, i.e., when the system (the memory and processing circuitry) calculates location of the target TR relative to the eyes, it can accurately calculate the location of the marker MR, based on this factory calibration information. At the same time, in this example, the cameras of the eye tracking device are tracking the visual axis of each eye, as well as the center of the limbus of each eye (tracked limbuses are depicted in FIG. 6B).

Dark blue graph G1 in FIG. 6A represents the distance between the centers of limbuses (Y axis) as a function of time (X axis). White graph G2 on the FIG. 6A represents the distance from the target TR to the mid point between two limbuses (Y axis) as a function of time (X axis). On the bottom graph in FIG. 6A, it is clearly seen that there are 3 times when the target is moved to the closest distance to the eyes and then moved further away. The top graph in FIG. 6A represents a subgraph of the bottom graph, emphasizing one of such moments. At some stage before the target distance graph G2 reaches its minimum, the distance between the centers of the limbuses (graph G1) stops decreasing and even starts growing. This is exactly the moment when the vergence breaks and patient fails to maintain binocular fixation on the target TR. The distance of the target TR to the eyes (or to the mid-point between the centers of the limbuses) is NPC. The vertical white line L in the top graph of FIG. 6A shows exactly this point.

In the following, some other aspects of the technique of the present disclosure are described, which relate to improvement of a cover test procedure for diagnostics of eyes alignment disorders.

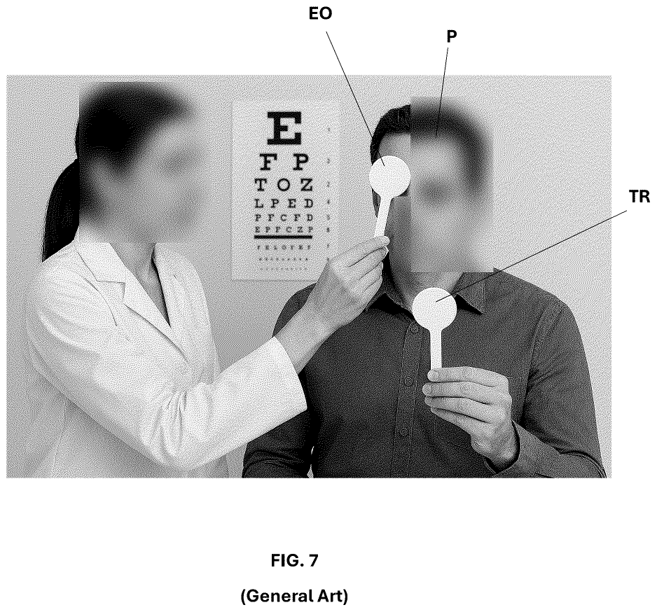

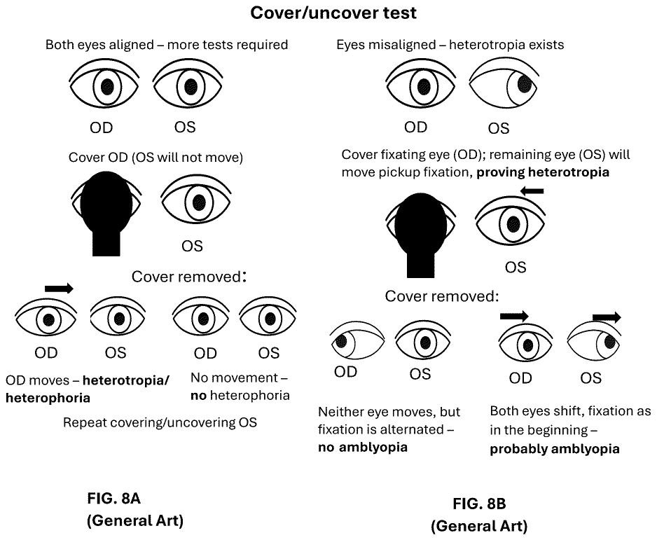

Reference is made to FIG. 7 and FIGS. 8A-8B. FIG. 7 is a general illustration of the cover test procedure: one eye OD (not seen) of a patient P is covered by an occluder EO, while the other eye OS is fixating on a target TR, which in the present example is held by the patient. It should be noted that the terms “OD” and “OS” are widely used to refer to the right (Oculus Dexter) and the left (Oculus Sinister) eyes, respectively. FIGS. 8A and 8B exemplify the cover/uncover tests to identify heterotropia/heterophoria condition (FIG. 8A) and also probability of amblyopia condition (FIG. 8B). Such cover/uncover test can generally be performed prior to alternate cover test.

The cover-uncover test is useful to identify a tropia and differentiate it from a phoria. The test is done by using an opaque or translucent occluder EO to cover one eye (see FIG. 7). The occluder EO is held (e.g., by a professional assistant, called here “examiner”) in front of the eye for a predetermined time period which is typically a few seconds, and then removed. FIGS. 8A and 8B show diagrams presenting two distinct clinical scenarios with their respective procedures and interpretations.

In the example of FIG. 8A, both eyes are initially aligned, i.e., the patient's eyes appear aligned on initial inspection. The examiner covers the right eye (OD) while the patient fixates on a target. The left eye (OS) is observed: it will not move since it was already fixating. The cover is then removed from the right eye OD. Possible outcomes upon removing the cover are: (1) the right eye OD moves to regain fixation, which is indicative of heterotropia condition (manifest misalignment) or heterophoria condition (latent misalignment that only appears when binocular vision is disrupted); (2) no movement of the right eye OD which is indicative of no heterophoria. The test is then repeated by covering and uncovering the left eye OS to check the left eye.

In the example of FIG. 8B, the eyes are visibly misaligned prior to the test (initial condition) which is indicative of the presence of heterotropia. The examiner covers the fixating right eye OD; the non-fixating left eye OS moves to pick up fixation, confirming heterotropia condition. The cover is then removed. Possible outcomes upon removing the cover are: (1) neither eye moves, but fixation is now with the left eye OS (alternated), this suggests no amblyopia, as the patient can maintain fixation with either eye equally; (2) both eyes shift and fixation returns to the right eye OD, which suggests probable amblyopia, as the brain strongly prefers one eye and does not maintain fixation with the weaker eye.

The above tests distinguish between manifest deviations (tropias) and latent deviations (phorias) and help assess whether a patient with strabismus has developed amblyopia based on their fixation preference. A strong, consistent preference for one eye suggests the non-preferred eye may be amblyopic.

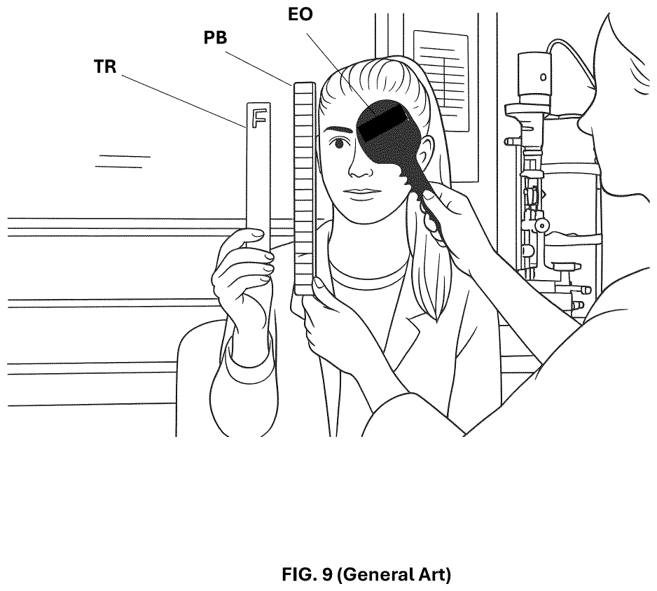

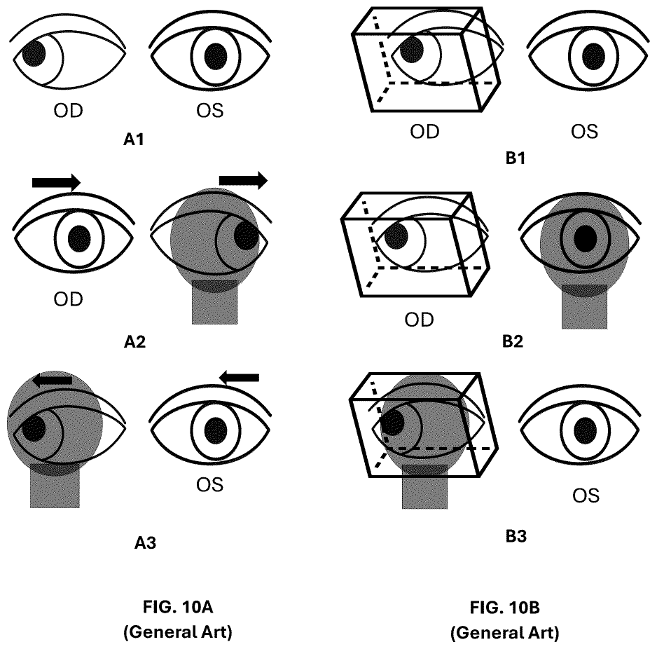

Reference is made to FIGS. 9 and 10A-10B illustrating the alternating cover test combined with prism measurement, used to quantify the angle of ocular deviation (strabismus).

FIG. 9 depicts the examination environment from the examiner's perspective. A patient is seated and fixating on a target TR: an eye chart, i.e., letter “F” is visible. The examiner holds an occlude/cover EO over the patient's left eye OS while a prism bar PB is properly accommodated in front of the patient's right eye OD (in this example, the patient holds the prism bar. The prism bar is a (handheld) device containing a series of prisms of progressively increasing strength. This setup allows the examiner to alternate the occluder between the patient's eyes and simultaneously introduce prisms of varying power until the deviation is neutralized.

The example of FIG. 10A (A1) shows exotropia (right eye is outward) with left eye fixating straight ahead. In FIG. 10A (A2) the occluder is placed over OS; both eyes shift slightly (conjugate movement) as OD takes up fixation (arrows show rightward movement); In FIG. 10A (A3) the occluder is placed over OD and as a result, the eyes shift back in the opposite direction (leftward arrows) as OS resumes fixation.

The examples of FIG. 10B show exotropia correction/neutralization using a prism of correct power. In FIG. 10B(B1) a prism (shown as a three-dimensional transparent block) is positioned in front of OD. The prism is being used to offset the underlying deviation (exotropia). When performing the alternate cover test, it is important to hold the occluder over each eye for at least a few seconds in order to allow the non-occluded eye enough time to pick up fixation. In general, the faster the eyes are able to recover when the occluder is switched, the better the control of the deviation.

In FIG. 10B (B2 and B3) the eyes do not move as the cover alternates from one eye to the other, i.e., when either OS is occluded (B2) while OD is behind the prism, or the occluder covers OD (behind the prism) while OS fixates on the target (B3). The goal of this test is to find the prism power that eliminates any refixation movement when alternating the cover. When the correct prism strength is reached, neither eye needs to move to pick up fixation because the prism optically compensates for the misalignment. This value, measured in prism diopters, quantifies the angle of deviation.

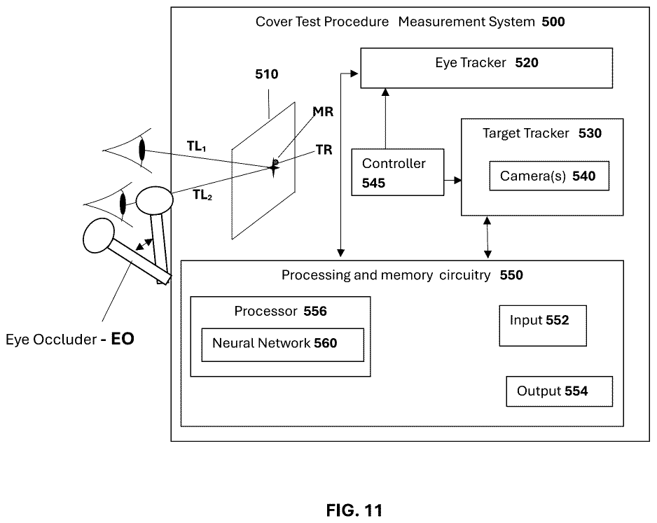

Reference is now made to FIG. 11 which shows schematically the configuration and operation of a measurement system 500 for eyes alignment measurements according to the principles of the present disclosure. The system 500 includes a target element/assembly 510, an eye tracking device 520, a target tracking unit 530, and a controller 545. The system 500 is associated with (i.e., including or connectable to) a processing and memory circuitry 550.

The target assembly 510 may generally be of any known suitable configuration. In some embodiments it may include a target TR (which may be in the form of a target pattern printed on a piece of cardboard) attached to a special optical marker MR. The target TR is used to fixate the patient's eyes on. During the various cover/uncover tests, target assembly 510 is configured and operable to measure angular deviations of the visual axis of each of the eyes relative to a theoretical fixation line extending from the respective eye to the fixation target, at a target distance from the respective eye in a range from 33 cm to 6 meters.

The eye tracking device 520 may generally be of any known suitable configuration. For example, the eye tracking device 520 may include an adjustable head-mounted apparatus with camera(s) in front of the patient's eyes. The eye tracking device 520 may be configured and operable to measure the position and orientation of the optical axis of each one of the patient's eyes with no required calibration.

The eye tracking device 520 may be configured and operable as described in the above-indicated U.S. Pat. Nos. 12,056,274 and 12,066,624, whose entire disclosures, except for any definitions, disclaimers, and inconsistencies, are incorporated herein by reference.

It should be noted that in some embodiments, system 500 may further include an automatic eye occluder (not shown in FIG. 11), configured and operable to automatically occlude a pre-selected eye of a patient. Such operation may be facilitated by positioning the patient's head against a chin rest equipment, such that eye occlusion can be guaranteed during automatic operation of the eye occluder (e.g., in the form of controllably automatically movable shutter-like element).

For the purposes of the present application, the eye tracking device 520 operates to perform first image acquisitions of each of the left and right eyes of a patient and provide measured eye-related data for each eye. The measured eye-related data is indicative of first time patterns of three-dimensional position and angular orientation of visual axes of the eyes during the cover test. The target tracking unit 530 is calibrated with respect to the eye tracking device 520 and operates to perform second image acquisitions of the optical marker (as described below) during the cover test and generate data indicative of a second time pattern of a three-dimensional spatial location of the fixation target during the cover test. The calibration between the devices 520 and 530 enables determination of the relative location of the eye tracker 520 and the target tracker 530. The eye tracking device 520 and the target tracking unit 530 operate synchronously to perform the first and second image acquisitions of, respectively, each eye and the target (via optical marker). To this end, the system 500 includes controller 545 which is configured and operable to control the synchronous image acquisitions. This enables the identification and use, in the data processing, of pairs of synchronously performed first and second image acquisitions, as described below.

It is known that the optical axis, being the symmetrical axis of the eye, is displaced from the visual axis which connects the target with the fovea. Since for the purposes of measuring misalignment parameters with cover tests, one needs to know amplitude and movement wavefront, displacement of the visual axis from the optical axis does not play a critical role, since both axes will move together. Thus, the continuous tracking of the optical axis may be easily transformed to respective tracking of the visual axis for the sake of these procedures.

The target TR is tracked by the target tracking unit 530 which includes at least one imager (camera) 540. Such camera may for example be a forward-facing camera mounted on the head-mounted apparatus of the eye tracker. The optical marker MR is attached to the target TR in such a way that it does not block the target TR which the patient's eyes follow with their gaze. As will be described further below, the optical marker may include a fiducial pattern enabling six-degree-of-freedom pose estimation.

The target tracking unit 530 includes at least one camera configured and operable to continuously track the relative target (stimulus) location relative to the eyes (while having no direct data about the relative location of the eyes with respect to the target TR). This can be facilitated by attaching the special marker MR to the target TR, providing that the marker MR is visible by the camera 540 during the whole measurement procedure. The target TR may be a small object, like a dot or a small letter. The eye tracking device 520 is configured and operable to measure the relative eyes'location in 3D and has no direct data about the relative target TR location.

The processing and memory circuitry 550 is configured and operable to be responsive to the data indicative of the first time pattern and data indicative of the second time pattern provided by the eye tracking device and the target tracking unit respectively, to determine, for each pair of the synchronously performed first and second image acquisitions, data indicative of angular deviation of the visual axis of each of the left and right eyes relative to a theoretical fixation line extending from the respective eye to the fixation target TR. Such theoretical fixation lines are shown in FIG. 11 as lines TL₁ and TL₂ in relation to the respective eyes. This enables obtaining data indicative of temporal dynamics of the angular deviations during the cover test and generating output data indicative thereof.

According to the present disclosure, the processing and memory circuitry 550 is capable of automatically and objectively measuring static and dynamic eyes misalignment during cover tests. This is provided by a continuous measurement of the position and orientation of the visual axis of each eye by the eye tracking device 520 and a continuous measurement of the target location by the target tracking unit 530.

When the eye of the patient is closed by an eye occluder EO that blocks the view of the target TR, it may deviate from the target—the stronger the deviation, the more severe may be the condition. The system 500 is configured and operable to accurately measure an amplitude and wavefront (dynamics) of the deviation, caused by covering the user's eye. The automatic and objective measurements with the eye tracker of the present disclosure make the tests much more accurate and repeatable, as compared to the currently practiced manual tests. Additionally, the measurements provide a recording of the dynamics of movement, thus allowing to store important data, beyond the single number of maximum deviation.

The processing and memory circuitry 550 is typically a computer system including inter alia data input and output utilities 552, 554, and a data processor 556. The measured data is provided, comprising first image data provided by the eye tracking device 520 and being indicative of the relative locations of the eyes and the position and orientation of the visual axes of the eyes, and second image data provided by the target tracking unit 530 and being indicative of relative locations of the target. The processing and memory circuitry 550 utilizes a pre-stored relative location of the eye tracking device 520 and the target tracking unit 530 to determine the location of the eyes and the target at any given moment of time when receiving data from both the target tracking unit 530 and the eye tracking device 520. The relative location of the eye tracking device 520 and the target tracking unit 530 may be factory pre-calibrated. Specifically, this calibration may be performed by showing the eye tracking device and target tracking unit cameras special markers with known relative location, thus enabling the calculation of the relative location of the eye tracker's and the target tracker's cameras by performing any known type of stereo cameras calibration procedure.

It should be noted that the image data may be indicative of the erroneous behavior of patients' eyes, which can be properly identified by the data processor (e.g., processor 556) to disregard the respective measured data. To this end, a neural network 560 can be used, which is properly learned and trained to identify such erroneous data.

The system 500 operates as follows: The specialist/operator (e.g., examiner) inputs or accesses patient's ID and other patient-related data such as interpupillary distance (IPD) and/or age and/or sex and/or special notes. Then, the head mounted device of the eye tracking device 520 is put on the patient's head and the alignment procedure is performed. During the alignment procedure, the specialist/operator adjusts the position of the eye tracking camera(s) to the patient's IPD and head, so that the camera(s) is/are located in the correct position before the patient's eyes. The patient is guided to help fine-tune alignment to the eye tracking channels. In this step, the patient receives corresponding instructions, and alignment is performed.

The patient is asked to fixate on the target TR. The practitioner may locate target TR on different distances from the patient's eyes. The system 500 measures accurate distance to TR at each location. The practitioner performs cover/uncover procedures and alternate cover procedures. The system 500 records eyes deviation amplitude and dynamics during these procedures—data that may be further used to help with the diagnostics and the whole data recording may be stored and become part of patient's electronic records. It is noted that the data indicative of the temporal dynamics may include saccadic velocity and acceleration profiles of the eyes during cover-uncover transitions of the eye condition.

The continuous measurement of the position and orientation of the visual axis of each eye by the eye tracking device 520 and the synchronized continuous measurement of the target location by the target tracking unit 530 provide sufficient information allowing the practitioner to continuously determine whether the patient fixates on the target with both eyes, when and to what extent (measured by angular deviation of the visual axis) any one of the visual axes is deviating from the theoretical fixation line described above. Specifically, eye movements and changes in fixation automatically determined by the technique of the present disclosure provide for an automatic diagnosis of heterotropia/heterophoria/amblyopia described above in relation to the various cover/uncover tests.

In embodiments utilizing an automatic eye occluder, a complete and automatic analysis of the cover test may be provided by the data processor 556 by attributing measured eyes deviation amplitudes and dynamics to the corresponding eye occlusion sessions.

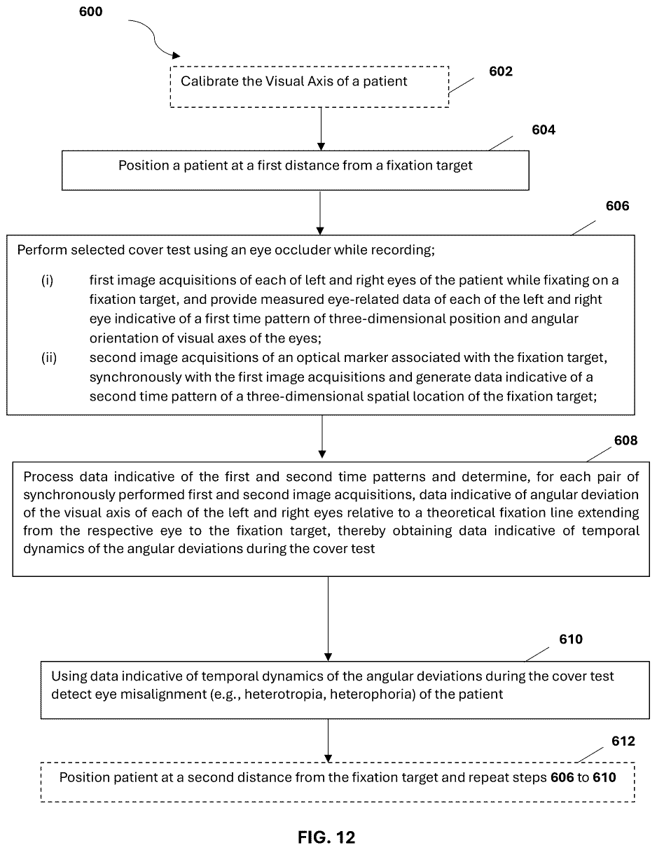

Reference is made to FIG. 12 exemplifying by a flow diagram 600 a method of the present disclosure for detecting eye misalignment of a patient. The method 600 may start with a one-time calibration step (step 602), i.e., calibration performed once for the patient's testing procedure, to determine the constant (per patient) displacement of the visual axis from the optical axis. Thus, the continuous tracking of the optical axes of the eyes provided by the eye tracking device 520 may be easily transformed into respective tracking of the visual axis, enabling measurement of the visual axes' locations in space. In step 604, the patient is positioned at a first distance from a fixation target. As mentioned above, it is important to perform the cover test at both near and distance. The practitioner selects a cover test (e.g., cover/uncover, alternate cover test) and uses an eye occluder (e.g., actuates operation of automatic occluder) to perform the selected cover test (step 606) while recording:
(i) first image acquisitions of each of left and right eyes of the patient while fixating on a fixation target, and provide first measured data, being eye-related data of each of the left and right eye indicative of a first time pattern of three-dimensional position and angular orientation of visual axes of the eyes; and
(ii) second image acquisitions of an optical marker associated with the fixation target, performed synchronously with the first image acquisitions and generate second measured data indicative of a second time pattern of a three-dimensional spatial location of the fixation target;

The data indicative of the first and second time patterns is processed in step 608 to determine, for each pair of synchronously performed first and second image acquisitions, data indicative of angular deviation of the visual axis of each of the left and right eyes relative to a theoretical fixation line extending from the respective eye to the fixation target. The obtained data is indicative of temporal dynamics of the angular deviations during the cover test. In step 610, the data indicative of temporal dynamics of the angular deviations during the cover test is used to detect eye misalignment (e.g., heterotropia, heterophoria) of the patient. In step 612, the patient may be positioned at a second distance from the fixation target and steps 606 to 610 may be repeated.

Thus, the technique of the present disclosure provides for automatically, and in some embodiments fully automatically, monitoring eye misalignment of a patient. As described above, the measured eye-related data of patient's eyes is first provided being indicative of visual axes of the patient's eyes relative to optical axis of the patient's eyes. Then, first and second measured data obtained during the cover test session(s) are provided. It should be noted that the processing of the so-obtained first and second measured data may be performed on-line, during the cover test, or off-line by accessing a storage device where the first and second measured data have been pre-stored. The first and second measured data are synchronously recorded during the selected cover test session on the patients' eye. The first measured data includes first image acquisitions of each of left and right eyes of the patient while fixating on the fixation target for at least one distance of the fixation target from the eye, and the second measured data includes second image acquisitions of the optical marker associated with the fixation target. The first and second measured data are processed to identify/determine data indicative of respective first time pattern of three-dimensional position and angular orientation of visual axes of the eyes and second time pattern of a three-dimensional spatial location of the fixation target. The first and second time patterns are then processed (utilizing the measured eye-related data of patient's eyes) and, for each pair of synchronously performed first and second image acquisitions, data indicative of angular deviation of the visual axis of each of the left and right eyes relative to a theoretical fixation line extending from the respective eye to the fixation target, is determined, where the angular deviation of the visual axis of each of the left and right eyes is indicative of temporal dynamics of the angular deviations during the cover test session. This enables detection of eye misalignment.

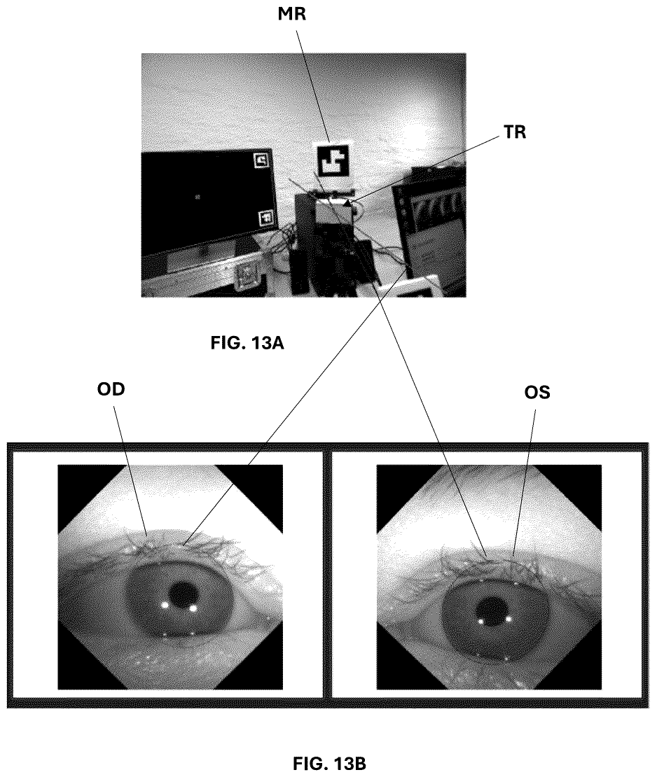

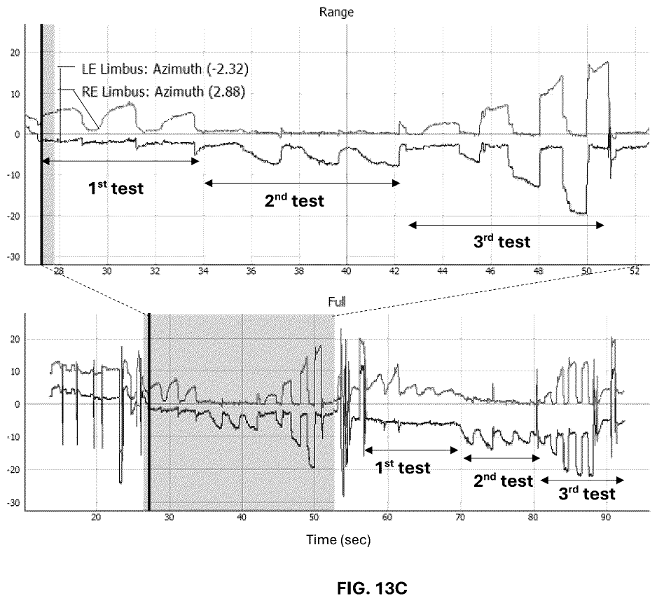

The results of exemplary recordings using the technique of the present disclosure are shown in FIGS. 13A to 13C. FIGS. 13A and 13B show the visual axes of the right (OD) and left (OS) eyes of a patient converging on a target TR which is a small dot. FIG. 13A also shows a special optical marker MR tracked by the camera 240 (not shown) during the whole measurement procedure FIG. 13C shows the recordings of deviations of the right eye (red curve) and the left eye (blue curve) during cover tests. The measured curves for the left and right eyes show the Azimuth, which is the deviation (in angular degrees) of the horizontal (X-axis) component of the gaze direction from the neutral (forward) direction. Thus, when any one of the curves shows a “wavy” motion, it indicates that the respective eye, being covered/occluded, is drifting sideways (rightwards or leftwards) and then “snaps” back when it is uncovered.

Three test sets are shown in an expanded time scale: in the first test set the right eye is covered/uncovered three times and it can be seen that while the right eye moves, the left eye remains almost static; in the second test set the left eye is covered/uncovered three times and it can be seen that while the left eye moves, the right eye remains almost static; in the third test set an alternate cover/uncover is performed where the cover is switched between left and right eye (alternating cover test). As a result, both eyes move, while movement amplitude is increasing, which is expected for a patient with heterophoria. The three test sets are repeated twice.

These results clearly demonstrate amplitude, as well as a dynamic behavior of occluded eyes while they deviate from the target.

1. A system for measuring near point of convergence (NPC) and/or convergence recovery point (CRP) of patient's eyes, the system comprising:
an eye tracking device configured and operable to provide measured eye-related data of the eyes being indicative of visual axes of the eyes associated with eyes' vergence during a measurement session;
a target tracking device, being calibrated with respect to the eye tracking device, and being configured and operable to monitor distances from the eyes to a moving target, onto which the eyes are required to be focused during said measurement session, and to provide measured target-related data indicative of a relative target location relative to the eyes; and
a memory and processing circuitry configured and operable to analyze the measured eye-related data and the measured target-related data and determine a relation between a change in the data indicative of the visual axes and data indicative of a change of said distance to the target during movement of said target during the measurement session, and to extract data indicative of said NPC and/or CRP from said relation.

2. The system according to claim 1, characterized by at least one of the following:
the memory and processing utility is configured and operable to analyze said measured eye-related data of the eyes and determine the change in the data indicative of visual axes of the eyes in space during the measurement session;
said data indicative of the visual axes comprises locations of the visual axes in space, said relation describing a mismatch between a point of intersection of said visual axes and the distance to a control location of the target corresponding to a condition of loss of focus of the eyes on said target;
said data indicative of the visual axes associated with the eyes'vergence comprises a distance between limbus centers of the eyes, said relation describing a change of said distance between the lumbus centers as a function of the change of said distance to the target, enabling to identify a value of the distance between the limbus centers corresponding to a predetermined change in said function, said value corresponding to eyes' vergence condition of loss of focus of the eyes on said target;
said measured eye-related data is image data.

3. The system according to claim 1, further comprising a target movement assembly comprising an elongated scale aligned with a rail on which the target is mounted for controlled movement along said rail, thereby enabling to monitor the distances from the subject's eyes to the target during said controlled movement of the target, a movement pattern of the target being calibrated with the eye-related data acquisitions pattern of the eye tracking device thereby enabling to determine said relation between the change in the data indicative of the visual axes associated with the eyes'vergence and the change of the distance to the target during said movement.

4. The system according to claim 1, wherein said measured eye-related data is image data, the eye tracking device having one of the following configurations:
the eye tracking device comprises at least one imaging device, each configured and operable to perform imaging of both eyes of the patient during the measurement session, thereby providing image data of the eyes being the measured eye-related data;
the eye tracking device comprises at least two imaging devices, associated with two eyes, respectively, such that at least one of said at least two imaging devices performs imaging of one of the two eyes and at least one other of the at last two imaging devices performs imaging of the other of the two eyes during the measurement session, thereby providing the measured eye-related data of the eyes;
the eye tracking device comprises at least two pairs of imaging devices, associated with two eyes, respectively, wherein each pair of the imaging devices performs imaging of the respective eye during the measurement session, thereby providing the measured eye-related data of the eyes.

5. The system according to claim 4, wherein the target tracking device has one of the following configurations:
the target tracking device comprises at least one imaging device each associated with the two eyes and configured and operable to perform imaging of the moving target and provide the measured target-related data, each of said at least one imaging device being located at calibrated distance from the one or more imaging devices of the eye tracking device;
the target tracking device comprises at least two imaging devices comprising at least one imaging device associated with one of the two eyes and at least one other imaging device associated with the other of the two eyes, said at least two imaging devices being configured and operable to perform imaging of the moving target and provide said measured target-related data, said at least two imaging devices being located at calibrated distances from the one or more imaging devices of the eye tracking device.

6. A method for measuring near point of convergence (NPC) and/or convergence recovery point (CRP) of patient's eyes, the method comprising:
providing measured eye-related data of the patient's eyes, indicative of visual axes of the eyes associated with eyes' vergence during a measurement session, and providing measured target-related data collected during the measurement session during which a target, onto which the eyes are required to be focused, is controllably moved with respect to the eyes, said measured target-related data being indicative of a relative target location relative to the eyes;
processing said measured eye-related data and said measured target-related data and determining a relation between a change in the data indicative of visual axes of the eyes and data indicative of a change of said distance to the target during movement of said target, and extracting data indicative of said NPC and/or CRP from said relation.

7. The method according to claim 6, characterized by at least one of the following:
said data indicative of the change of the distance to the target corresponds to the movement of the target from a larger distance towards the eyes, thereby enabling to extract the data indicative of the NPC;
said data indicative of the change of the distance to the target corresponds to the movement of the target from a relatively close distance towards further away from the eyes, thereby enabling to extract the data indicative of the CRP.

8. The method according to claim 6, wherein said processing comprises: analyzing the measured eye-related data and determining the change in said data indicative of the visual axes in space during the measurement session.

9. The method according to claim 6, characterized by one of the following:
said data indicative of the visual axes comprises locations of the visual axes in space, said relation describing a mismatch between a point of intersection of said visual axes and the distance to a control location of the target corresponding to a condition of loss of focus of the eyes on said target;
said data indicative of the visual axes associated with the eyes'vergence comprises a distance between limbus centers of the eyes, said relation describing a change of said distance between the lumbus centers as a function of the change of said distance to the target, enabling to identify a value of the distance between the limbus centers corresponding to a predetermined change in said function, said value corresponding to eyes' vergence condition of loss of focus of the eyes on said target.

10. The method according to claim 6, wherein said data indicative of the visual axes associated with the eyes'vergence comprises a distance between limbus centers of the eyes, said relation describing a change of said distance between the lumbus centers as a function of the change of said distance to the target comprises a decrease in said distance between the lumbus centers as the target is moved from a larger distance towards the eyes, data indicative of a stopped decrease in said distance between the lumbus centers as the target is moved from a larger distance towards the eyes is indicative of NPC.

11. The method according to claim 6, wherein said data indicative of the visual axes associated with the eyes'vergence comprises a distance between limbus centers of the eyes, said relation describing a change of said distance between the lumbus centers as a function of the change of said distance to the target comprises an increase in said distance between the lumbus centers as the target is moved from a relatively close distance towards further away from the eyes, data indicative of a gradual increase in said distance between the lumbus centers as the target is moved towards further away from the eyes is indicative of CRP.

12. The method according to claim 6, wherein said measured eye-related data is image data.

13. The method according to claim 12, wherein said providing of the measured eye-related data comprises one of the following: performing image acquisitions by at least one imaging device, each associated with two eyes, and providing said measured eye-related data; and performing image acquisitions by at least two imaging devices associated with two eyes, respectively, and providing said measured eye-related data.

14. The method of claim 6, wherein said providing of the measured target-related data comprises imaging the target while said target is moved along a rail aligned with an elongated scale, and monitoring the distances from the subject's eyes to the target during the movement of the target, a movement pattern of the target being calibrated with the measured eye-related data acquisitions pattern.

15. The method according to claim 12, wherein said providing of the measured target-related data comprises one of the following: imaging of the moving target by at least one imaging device, each associated with the two eyes, each of said at least one imaging device being located at calibrated distance from one or more imaging devices of the eye tracking device; and imaging of the moving target by at least two imaging devices associated with the two eyes, respectively, said two imaging devices being located at calibrated distances from the respective two pairs of the imaging devices of the eye tracking device.

16. A system for performing a cover test to detect eye misalignment of a patient, the system comprising:
a target assembly comprising a fixation target exposed to be visible to the patient, and an optical marker associated with the fixation target;
an eye occluder operable to selectively block and unblock vision of one of the left and right eyes of the patient, such that said one of the eyes sequentially experiences cover-uncover conditions during the cover test;
an eye tracking device configured and operable to perform first image acquisitions of each of left and right eyes of the patient and provide measured eye-related data of each of the left and right eyes, said measured eye-related data being indicative of a first time pattern of three-dimensional position and angular orientation of visual axes of the eyes during the cover test;
a target tracking unit being calibrated with respect to the eye tracking device, the target tracking unit being configured and operable to perform second image acquisitions of said optical marker during the cover test synchronously with the first image acquisitions performed by the eye tracking device, and generate data indicative of a second time pattern of a three-dimensional spatial location of the fixation target during the cover test;
a processing and memory circuitry configured and operable to carry out the following: receive and process said data indicative of the first time pattern and said data indicative of the second time pattern, and determine, for each pair of synchronously performed first and second image acquisitions, data indicative of angular deviation of the visual axis of each of the left and right eyes relative to a theoretical fixation line extending from the respective eye to said fixation target, thereby obtaining data indicative of temporal dynamics of said angular deviations during the cover test; and generate output data indicative thereof.

17. The system according to claim 16, wherein the optical marker comprises a fiducial pattern enabling six-degree-of-freedom pose estimation.

18. The system according to claim 16, configured and operable to measure said angular deviations of the visual axis of each of the eyes relative to said theoretical fixation line at a target distance from the respective eye in a range from 33 cm to 6 meters.

19. The system according to claim 16, wherein said data indicative of temporal dynamics comprises saccadic velocity and acceleration profiles of the eyes during cover-uncover transitions of the eye condition.

20. The system according to claim 16, wherein said processing and memory circuitry is configured and operable to apply neural network processing to said first and second time patterns to identify and exclude erroneous eye tracking data caused by blinks or artifacts.

21. A method for monitoring eye misalignment of a patient, the method comprising:
providing measured eye-related data of patient's eyes, indicative of visual axes of the patient's eyes relative to optical axis of the patient's eyes;
providing first and second measured data, synchronously recorded during a selected cover test session on the patients' eyes, comprising first image acquisitions of each of left and right eyes of the patient while fixating on a fixation target for at least one distance of the fixation target from the eye, and second image acquisitions of an optical marker associated with the fixation target;
processing said first and second measured data and determining data indicative of respective first time pattern of three-dimensional position and angular orientation of visual axes of the eyes and second time pattern of a three-dimensional spatial location of the fixation target; and
utilizing the measured eye-related data of patient's eyes and processing the first and second time patterns and determining, for each pair of synchronously performed first and second image acquisitions, data indicative of angular deviation of the visual axis of each of the left and right eyes relative to a theoretical fixation line extending from the respective eye to the fixation target, said angular deviation of the visual axis of each of the left and right eyes being indicative of temporal dynamics of the angular deviations during the cover test session, enabling detection of eye misalignment.

22. The method according to claim 21, where said selected cover test includes at least one of the following: cover/uncover test and alternate cover test.

23. The method according to claim 21, wherein said providing of the first and second measured data comprises performing the cover test session by operating a system comprising:
a target assembly comprising a fixation target exposed to be visible to the patient, and an optical marker associated with the fixation target;
an eye occluder operable to selectively block and unblock vision of one of the left and right eyes of the patient, such that said one of the eyes sequentially experiences cover-uncover conditions during the cover test;
an eye tracking device configured and operable to perform the first image acquisitions of each of left and right eyes of the patient and provide the first measured data of each of the left and right eyes, indicative of the first time pattern of three-dimensional position and angular orientation of visual axes of the eyes during the cover test session;
a target tracking unit being calibrated with respect to the eye tracking device, the target tracking unit being configured and operable to perform the second image acquisitions of said optical marker during the cover test synchronously with the first image acquisitions and generate data indicative of the second time pattern of a three-dimensional spatial location of the fixation target during the cover test.
